(19) United States
(12) Patent Application Publication
(10) Pub. No.: US 2022/0111101 A1
(43) Pub. Date: Apr. 14, 2022

(54) METHOD AND SYSTEM FOR DECONTAMINATING AN ENVIRONMENT BY MEANS OF OZONE

(71) Applicant: SALL S.R.L., Cavriago (IT)

(72) Inventor: Marco Luigi Tassi, Montù Beccaria (PV) (IT)

(73) Assignee: SALL S.R.L., Cavriago (IT)

(21) Appl. No.: 17/398,768

(22) Filed: Aug. 10, 2021

(30) Foreign Application Priority Data
Oct. 13, 2020 (IT) 102020000024085

(51) Int. Cl. A61L 9/015; F24F 3/16; A61L 9/20; F24F 3/14

(52) U.S. Cl. CPC A61L 9/015; F24F 3/16; A61L 9/20; F24F 3/14; A61L 2209/16; A61L 2209/111; A61L 2209/14; F24F 2221/56; A61L 2209/212

(57) ABSTRACT

A method for decontaminating an environment includes measuring an ozone concentration value present inside the environment and repeatedly carrying out a sequence of steps comprising: increasing the quantity of ozone present in the environment until the measured ozone concentration value reaches a first value and lowering the quantity of ozone present in the environment until the measured ozone concentration value reaches a second value lower than the first value.

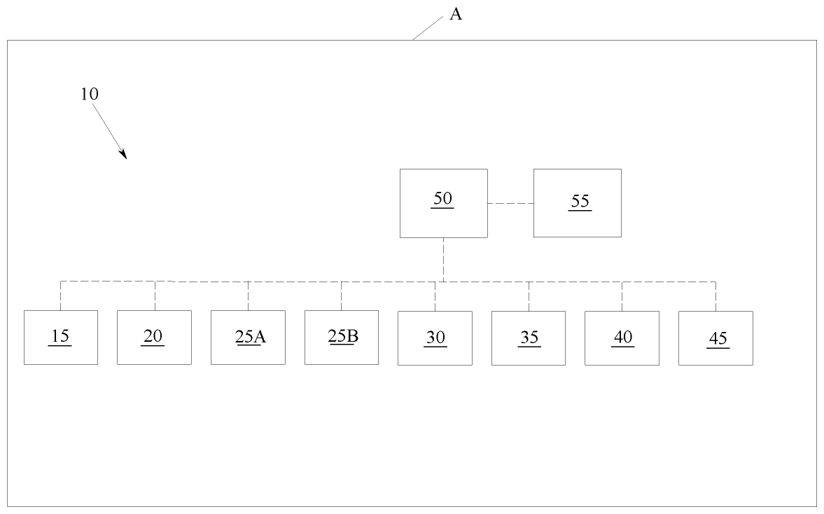

### CROSS-REFERENCE TO RELATED APPLICATION

This patent application claims the benefit of priority to Italian Patent Application No. 102020000024085, filed Oct. 13, 2020, and entitled, “Method and Device for Decontaminating an Environment by Means of Ozone,” which is incorporated in its entirety herein by this reference.

### TECHNICAL FIELD

The present disclosure relates to the field of decontamination of environments from pathogens such as viruses, bacteria and spores, and from unwanted chemical compounds. In particular, the disclosure relates to a method and a system for decontaminating an environment by means of ozone.

### BACKGROUND

In the field of the treatments for decontaminating environments, the use of ozonizers, i.e. devices capable of generating ozone starting from oxygen, is known. These ozonizers are generally arranged inside an environment to be sanitized and configured to deliver ozone until a certain desired concentration is reached in order to eliminate pathogens such as viruses, bacteria or spores, i.e. in order to inhibit the ability thereof to replicate. Although theoretically this procedure should be sufficient to achieve a desired high level of decontamination, field analyses have highlighted that, in a substantially equivalent way to what happens with the use of antibiotics, the pathogens, as they are exposed to a same concentration of ozone, become resistant thereto.

It will be appreciated that this background description has been provided to aid the reader, and is not to be taken as an indication that any of the indicated problems were themselves appreciated in the art. While the described principles can, in some aspects and embodiments, alleviate the problems inherent in other systems, it will be appreciated that the scope of the protected innovation is defined by the attached claims, and not by the ability of any disclosed feature to solve any specific problem noted herein.

### SUMMARY

The present disclosure, in one aspect, is directed to embodiments of a method of decontaminating an environment. In embodiments, a method of decontaminating an environment includes measuring an ozone concentration value present in the environment and carrying out a sequence of steps comprising: (i) increasing the quantity of ozone present in the environment until the measured ozone concentration value reaches a first value, and (ii) lowering the quantity of ozone present in the environment until the measured ozone concentration value reaches a second value lower than the first value. The sequence of steps (i) and (ii) is repeated at least once such that the measured ozone concentration inside the environment sequentially reaches the first value, the second value, again the first value, and again the second value.

In another aspect, the present disclosure is directed to embodiments of a system for decontaminating an environment. In one embodiment, a system for decontaminating an environment includes an ozone generator, an ozone sensor, and an electronic control unit. The ozone generator device is configured to introduce ozone into the environment. The ozone sensor is configured to measure an ozone concentration value inside the environment. The electronic control unit is operatively connected to the ozone sensor and to the ozone generator device. The electronic control unit is configured to selectively activate the ozone generator device to alternatingly (i) deactivate the ozone generator device when the ozone concentration value measured by the ozone sensor reaches a first value and (ii) activate the ozone generator device when the ozone concentration value measured by the ozone sensor reaches a second value lower than the first value.

Further and alternative aspects and features of the disclosed principles will be appreciated from the following detailed description and the accompanying drawings. As will be appreciated, the methods and systems disclosed herein are capable of being carried out in other and different embodiments, and capable of being modified in various respects. Accordingly, it is to be understood that both the foregoing general description and the following detailed description are exemplary and explanatory only and do not restrict the scope of the appended claims.

### BRIEF DESCRIPTION OF THE DRAWINGS

Further features and advantages of the invention will be more apparent after reading the following description provided by way of a non-limiting example, with the aid of the figure.

The Figure is a schematic view of an exemplary embodiment of a system for carrying out a method according to the invention.

### DETAILED DESCRIPTION OF EXEMPLARY EMBODIMENTS

In embodiments, a method of decontaminating an environment following principles of the present disclosure includes measuring an ozone concentration value present in the environment; and carrying out a sequence of alternating steps comprising: increasing the quantity of ozone present in the environment until the measured concentration value reaches a first value, and lowering the quantity of ozone present in the environment until the measured concentration value reaches a second value lower than the first value. Embodiments of a method for decontaminating an environment following principles of the present disclosure can, by means of ozone regulation, decrease the possibility that pathogens become resistant to ozone.

In embodiments, the method comprises carrying out the aforesaid sequence of steps two or more times so that the ozone concentration follows a trend over time that can be represented by a continuous graph, for example with an oscillatory or sinusoidal trend over time. In embodiments, the sequence of steps is carried out such that the continuous graph includes in succession at least a first rising edge up to the first value and a subsequent falling edge to the second value, followed by at least a second rising edge, starting from the second value reached by the first falling edge again at the first value, and by a subsequent second falling edge, starting from the first value reached by the second rising edge again at the second value, and so on.

In embodiments, the method comprises continuously varying the ozone concentration inside the environment so as to consecutively (i.e. in the same temporal sequence and in continuous succession within it) reach the first value, reach the second value, reach again the first value, reach again the second value, and so on. In embodiments, the variation of the ozone concentration value between the first value and the second value can be carried out several times (for example even more than twice) by consecutively repeating the aforesaid sequence of steps. The repeated (at least twice) variation of the concentration between the first value and the second value can have the effect that the parasites are unable to adapt to the environmental conditions and to develop the resistance highlighted in the aforesaid field tests, making decontamination particularly effective.

In another aspect of the invention, the step of increasing the quantity of ozone can comprise introducing ozone by means of an ozone generator device. In embodiments, the method including increasing the quantity of ozone by means of an ozone generator device allows the ozone concentration to be increased in a particularly rapid way.

In still another aspect of the invention, a method following principles of the present disclosure includes the step of lowering the quantity of ozone by introducing ultraviolet (UV) rays into the environment. In embodiments, a method following principles of the present disclosure including the step of lowering the quantity of ozone inside the environment and therefore of the concentration thereof by introducing UV rays into the environment can be advantageously accelerated, making this effect of continuous variation of the concentration even more effective and preventing pathogens from developing a resistance.

A further aspect of the invention envisages that the step of lowering the quantity of ozone can comprise filtering the air present in the environment by means of a filter suitable for converting the ozone into other substances. Also this solution allows the quantity/concentration of ozone to be lowered in a particularly rapid way, and therefore the possibility of development resistance to ozone by pathogens to be obviated. In embodiments, the filter suitable for converting the ozone into other substances can be an activated carbon-based filter or a manganese oxide- and copper oxide-based filter, for example based on the catalyst known under the trade name of Carulite 200. In embodiments, the step of lowering the quantity of ozone by filtering the air present in the environment by means of a filter suitable for converting the ozone into other substances can make it possible to convert ozone into oxygen, i.e. into substances already present in the environment without substantially altering the environmental conditions.

In still another aspect, a method following principles of the present disclosure can further comprise: measuring a level of humidity present in the environment; comparing the measured level with a reference value; generating an alarm signal and/or humidifying the environment if the measured level of humidity is lower than said reference value. In at least some of such embodiments, the method allows controlling the environment to be sanitized in a particularly effective way before proceeding with the decontamination action so as to be able to take any countermeasures to maximize the effectiveness of ozone on pathogens. In fact, if the level of humidity were too low, the ozone may be ineffective in acting on pathogens.

In a further aspect, a method following principles of the present disclosure can further comprise: measuring a value of a temperature inside the environment; comparing the measured value with a threshold value; generating an alarm signal and/or lowering the temperature of the environment if the measured value is greater than said threshold value. In at least some of such embodiments, the method allows controlling the environment to be sanitized in a particularly effective way before proceeding with the decontamination action, so as to be able to take any countermeasures to maximize the effectiveness of ozone on pathogens. In fact, with environment temperatures that are too high (particularly above 40° C.) the ozone can rapidly decays to the state of oxygen making it substantially difficult to reach the necessary concentration of ozone in the environment.

In still a further aspect, a method following principles of the present disclosure can further comprise generating an alarm signal and/or interrupting the ozone introduction step if, after a predetermined maximum time has lapsed from the beginning of said introduction step, the measured concentration value is still lower than said first value. In at least some of such embodiments, the method allows controlling the decontamination action in a particularly effective way, avoiding waste in terms of energy consumption and resources in general. In fact, by monitoring the time lapsed, the quantity of ozone delivered and the value of the ozone concentration, the method basically allows to check if the necessary level of concentration can be reached and, if not, to signal it through the alarm signal.

Another aspect of the invention envisages that the second concentration value can be in a range between ten percent (10%) and twenty-five (25%) of the first value. In at least some of such embodiments, there is a difference between the first value and the second value such as to increase the effectiveness of the decontamination work performed.

In another aspect of the disclosure, embodiments of a system for decontaminating an environment are provided. In embodiments, a system for decontaminating an environment comprises: an ozone generator device configured to introduce ozone into the environment; a probe configured to measure an ozone concentration value inside the environment; and an electronic control unit operatively connected to the probe and to the ozone generator device, the electronic control unit being configured to repeatedly deactivate the ozone generator device, when the concentration value measured by the sensor reaches a first value, and to activate the ozone generator device when the concentration value measured by the sensor reaches a second value lower than the first value.

In at least some of such embodiments, the invention makes available a system capable of delivering ozone and regulating the quantity delivered in a variable manner so as to maximize the effectiveness of the decontamination work.

In another aspect, embodiments of the system can comprise a temperature sensor operatively connected to the electronic control unit and configured to measure a temperature value present in the environment.

In at least some embodiments, the system is able to control at least one parameter indicative of a condition of the environment to be sanitized that might influence the effectiveness of the ozone against pathogens, so as to be able, if necessary, to take appropriate countermeasures.

In still another aspect, embodiments of a system constructed according to principles of the present disclosure can comprise a hygrometric sensor operatively connected to the electronic control unit and configured to measure a humidity value present in the environment. In at least some embodiments, the system is able to control a parameter indicative of a condition of the environment to be sanitized that might influence the effectiveness of the ozone against pathogens, so as to be able, if necessary, to take appropriate countermeasures.

In a further aspect, embodiments of a system constructed according to principles of the present disclosure include an electronic unit that is operatively connected to an interface and configured to generate an alarm signal through it. In at least some of such embodiments, the system is equipped with an interface that can allow interacting with an operator assigned to commissioning the plant, for example, in order to signal anomalies or alarms relating to environmental conditions that are critical for the decontamination effectiveness.

With particular reference to the Figure, the number 10 globally indicates a system for decontaminating an environment A (such as, e.g., in a plant), particularly for decontaminating the air of said environment A and of all the surfaces present inside it. The environment A is preferably a closed or circumscribed environment.

It is specified that decontamination means an operation aimed at the reduction by removing and/or killing and/or inactivating bacteria, viruses, fungi, protozoa, spores, and at the elimination of unwanted chemical compounds in order to control/cancel the risk of infection for people or of contamination of objects or environments.

Particularly, in the modalities that will be described below, the system 10 is configured to introduce ozone into the environment A in order to carry out the aforesaid decontamination.

The system 10 includes an ozone generator device 15 configured to generate ozone starting from oxygen and to introduce the generated ozone into the environment A.

The ozone generator device 15 includes at least one so-called ozone-producing cell, for example of the corona effect type. Such ozone-producing cells are known to the person skilled in the art and therefore are not described in detail.

The ozone-producing cell has an inlet through which it is adapted to receive a gas flow containing at least a quantity of oxygen and an outlet through which the gas flow outflows. The gas flow, while running through the ozone-producing cell from the inlet to the outlet, is made to pass through an interspace defined between a central electrode and a tube made of dielectric material coated with a conductive film, one of which is supplied by a suitable power supply voltage (for example a power supply voltage comprised between 3000 and 8000 V and at a frequency between 18 and 30 KHz) while the other one is suitably connected to the ground. In this way, the so-called corona effect is generated (at the interspace), which is able to convert at least a part of the oxygen contained in the gas flow running through the ozone-producing cell into ozone.

If the gas flow consists of pure oxygen, the ozone generator device 15 can comprise an oxygen tank, for example an oxygen cylinder or an oxygen concentrator, connected sealingly to said inlet of the ozone-producing cell and adapted to the supply thereof.

Alternatively, the gas flow can comprise air, possibly suitably filtered and/or treated. In this case, the ozone generator device 15 can comprise a suction unit configured to withdraw a certain quantity of air from the outside, for example from the environment A, and introduce it into the ozone-producing cell by means of a supply duct which includes a suitable air filtration arrangement.

Regardless of this, the ozone generator device 15 can comprise a delivery duct connected sealingly to the outlet of the ozone-producing cell, and open onto the environment A by means of a delivery mouth.

The ozone generator device 15 can also comprise a diffuser associated with said delivery mouth of the delivery duct which is configured to diffuse (i.e. convey in a plurality of different directions) the ozone generated by the ozone-producing cell at the outlet from the delivery mouth thereof. For example, said diffuser can comprise a fan or impeller the rotation of which is driven by a suitable drive motor.

The plant 10 can then comprise a filtration arrangement 20 configured to filter (if necessary) the air present in the environment A, in order to reduce the ozone concentration. The filtration arrangement 20 can comprise a suction device of the known type, for example comprising a suction fan or impeller and a drive motor (for example an electric motor) adapted to set said fan or impeller in rotation so as to draw a certain quantity of air to be filtered in the unit of time.

The filtration arrangement 20 can comprise a conveying system configured to receive said quantity of air withdrawn from the environment A, to make it pass through a suitable filter, and to introduce a corresponding quantity of filtered air back into environment A. For example, the conveying system can comprise a conveying tube equipped with an inlet mouth for the air to be filtered and with an outlet mouth for the filtered air.

The filter can be connected to the inlet mouth of the conveying tube or to the outlet mouth, or it can be inserted inside the conveying tube. Particularly, said filter of the filtering arrangement 20 can be a filter adapted to convert the ozone present in the quantity of sucked air into other substances.

For example, said filter can be an activated carbon-based filter that allows the transformation of the ozone into carbon dioxide. Alternatively, said filter can be a manganese (di)oxide- and copper oxide-based filter, for example. In embodiments, a commercially-available catalyst can be used which allows the conversion of ozone into oxygen, such as those known under the trade name of Carulite 200 from Carus Corp. of Peru, Ill.

The plant 10 can further comprise a humidifier device 25A arranged inside the environment A and configured to vary (raise) a level of humidity present inside the environment A, for example by introducing a water vapor into the environment A.

In addition or alternatively, the plant 10 can comprise a (temperature) conditioner device 25B of the air contained inside the environment A for cooling thereof (for example already present and installed inside the environment A).

The plant 10 may further comprise a UV (ultraviolet) rays source 30 arranged inside the environment A to be sanitized. Particularly said UV rays source 30 can be of the LED (light emitting diode) type. In embodiments, the source 30 is preferably configured to introduce UVC rays into the environment A to be sanitized, whereby UVC refers to ultraviolet rays characterized by a wavelength in a range between 100 nm and 280 nm. Preferably, the source 30 is configured to introduce UVC rays characterized by a wavelength of 254 nm into the environment A.

The system 10 can include a probe 35 adapted to be arranged inside the environment A to be sanitized and configured to measure an ozone concentration value inside the environment A, i.e. configured to generate a first signal indicative of an ozone concentration value present in the environment A.

The system 10 can also comprise a temperature probe 40 adapted to be arranged inside the environment A to be sanitized and configured to measure a temperature value present inside the environment A, i.e. configured to generate a second signal indicative of a temperature value present in the environment A.

The system 10 can also comprise a hygrometric probe 45 adapted to be arranged inside the environment A to be sanitized and configured to measure a humidity value present inside the environment A, i.e. configured to generate a third signal indicative of a humidity value present in the environment A.

In some embodiments, the ozone concentration probe 35, the temperature probe 40, and/or the hygrometric probe 45 can be integrated into a single device called environmental probe.

The system 10 can further comprise an electronic control unit 50 which is configured to manage the operation of the system 10 automatically and/or semi-automatically, for carrying out a method for decontaminating the environment A. The electronic control unit 50 is, for example, of the type comprising a processor, equipped with a memory unit, in which a program is stored and which commands the operating steps of the decontamination method.

The electronic control unit 50 is operatively connected, by means of cables or possibly by wireless technology, to each electrically/electronically controllable component of the plant 10, for the control and command of the operation thereof. For example, the electronic control unit 50 is operatively connected to the ozone generator device 15 and, if present, to the filtration arrangement 20, to the humidifier device 25A, to the conditioner device 25B, to the UV rays source 30, to all the probes 35, 40, 45 and any further sensors of the plant 10.

In particular, the electronic control unit 50 can be operatively connected to the probe 35 to receive the first signal and to detect and/or acquire, on the basis of said first signal, the ozone concentration value in the environment A. Furthermore, the electronic control unit 50 can be operatively connected to the temperature probe 40 to receive the second signal and to detect and/or acquire, on the basis of said second signal, the temperature value in the environment A. Furthermore, the electronic control unit 50 can be operatively connected to the hygrometric probe 45 to receive the third signal and to detect and/or acquire, on the basis of said third signal, the humidity value present in the environment A.

The electronic control unit 50 can also be operatively connected to an interface 55, through which it can output some operating parameters of the system 10, for example the temperature value measured by means of the temperature probe 40 and/or the humidity value measured by means of the hygrometric probe 45 and/or the ozone concentration value measured by means of the probe 35.

In particular, the electronic control unit 50 can be configured to generate an alarm signal (transmitted and emitted) by means of the interface 55, for example when it detects an operating anomaly of the plant 10 and/or a critical temperature value, i.e. a temperature value equal to or greater than a threshold value that is preset in the electronic control unit 50, and/or a critical humidity value, i.e. a humidity value equal to or lower than a reference value that is preset in the electronic control unit 50, or other critical value detected. The alarm signal can be, for example, an acoustic and/or visual signal and/or any other type.

The electronic control unit 50 could also be configured to receive some operating parameters as input, by means of said interface 50. In particular, the electronic control unit 50 can be configured to receive as input, by means of the aforesaid interface 55, a maximum time value of continuous activation of the ozone generator device 15, and a value of a volume of the environment A to be sanitized.

For example, the interface 55 can comprise or consist of the monitor of a PC or be of the type of a mobile device provided to an operator assigned to the plant 10 or consists of a panel of indicators and warning lights. In other embodiments, the electronic control unit 50 and/or the interface 55 could be integrated into the ozone generator device 15.

The operation of the system 10, which is preferably commanded and controlled by the electronic control unit 50, allows to carry out a method for decontaminating the environment A which is described below.

In embodiments, the method includes measuring (in order to monitor substantially continuously) an ozone concentration value present inside the environment A, for example by means of the probe 35 operatively connected to the electronic control unit 50.

While this monitoring is being carried out, the method includes increasing the quantity of ozone present in the environment A until the measured concentration value reaches a first value, for example by means of the ozone generator device 15. This first value can be supplied to the electronic control unit 50 by the user, for example through the interface 55, or it can be determined by the electronic unit 50 thereof, for example on the basis of a preset program and/or other input parameters, for example on the basis of the decontamination level to be obtained.

After reaching the first value (or in any case a value not lower than the first value), the method includes lowering the quantity of ozone present in the environment A until the measured concentration value reaches a second value lower than the first value. For example, the second value can be comprised between 10% and 25% of the first value. This second value can also be supplied to the electronic control unit 50 by the user, for example through the interface 55, or it can be determined by the electronic unit 50 thereof, for example on the basis of a preset program and/or other input parameters, for example on the basis of the decontamination level to be obtained.

This lowering of the ozone concentration can be achieved either spontaneously or naturally, simply by waiting the time necessary for the ozone to reconvert, or in a forced way, for example by activating the filtration arrangement 20, in order to filter the air and convert the ozone present therein into other substances (carbon dioxide or oxygen), and/or by activating the UV rays source 30, so as to introduce UV rays into the environment A which accelerate the decay of the ozone to the state of oxygen.

Once the second concentration value has been reached (or in any case a value not higher than the second value), the method includes increasing the ozone concentration again up to at least the first value and, subsequently, lowering again the ozone concentration up to at least the second value, in the same way as outlined above.

This sequence of steps, i.e. the raising of the ozone concentration up to the first value and the subsequent lowering of the ozone concentration up to the second value, can then be repeated countless times until the full decontamination of environment A.

In practice, the method comprises carrying out the aforesaid sequence of steps two or more times, so that the ozone concentration follows a trend over time that can be represented by means of a continuous graph, for example oscillatory or sinusoidal, which includes in a temporal succession at least a first rising edge up to the first value and a (substantially immediately) subsequent falling edge to the second value, as well as at least a second rising edge to the first value, starting from said second value (substantially immediately) subsequent to the first falling edge and a second falling edge to the second value, starting from the first value (substantially immediately) subsequent to the second rising edge. In embodiments, the method can include continuously varying the ozone concentration inside the environment A in order to consecutively reach the first value, reach the second value, reach the first value again, reach the second value again, and so on. It is hereby specified that the term “consecutively” means substantially immediately subsequent temporally, i.e. in the same temporal sequence and in continuous succession within it.

For example, in embodiments, the electronic control unit 50 can be configured to repeatedly activate the ozone generator device 15 when the concentration value measured by the sensor reaches the second value lower than the first value and to deactivate the ozone generator device 15 when the concentration value measured by the sensor reaches a first value. This ensures that the ozone concentration always reaches the first and second values.

However, due to inevitable “inertia” in the concentration variation, these values might be exceeded, causing excessive and substantially useless consumption of ozone. In particular, even after the ozone generator 15 has been switched off, the ozone concentration in the environment A could continue to increase spontaneously for a certain time, reaching values higher than the first value, before starting to decrease. Similarly, even after the ozone generator 15 has been switched on again and the reduction means (e.g. filtration arrangement 20 and/or UV rays source 30) have been switched off, the ozone concentration in the environment A could continue to decrease spontaneously for a certain time, reaching values lower than the second value, before starting to increase again.

Furthermore, a control based solely on the measurement of the ozone concentration might be greatly influenced by the positioning of the probe and by any random events, such as for example by the fact that the probe is hit by an air flow having locally an ozone concentration different from the global concentration present in the environment A to be sanitized. To obviate these contraindications, in embodiments, the electronic control unit 50 is configured to perform, for example on the basis of a digital analysis of data coming from the probes and/or other data, a predictive modelling of the ozone concentration trend in the environment A, so as to also foresee the aforesaid “inertia” and/or neglect “temporary or localized events,” thus allowing the operation of the ozone generator 15 to be interrupted and resumed at the most appropriate times so that the ozone concentration in the environment A is kept substantially within a range between the first and second values.

In addition to what has been described so far, the method can include, in embodiments, generating an alarm signal, for example by means of the interface 55 operatively connected to the electronic control unit 50, and/or interrupting the ozone introduction step, for example by de-activating the ozone generator device 15, if, after a predetermined maximum time from the start of said introduction step has lapsed (for example the maximum activation time of the ozone generator device 15), the measured concentration value is still lower than said first value. In embodiments, the aforesaid maximum time can for example be preset in the electronic control unit 50.

The method can also include measuring a level of humidity present in the environment A, comparing the level of humidity measured with a reference value, and generating an alarm signal and/or humidifying the environment A if the measured level of humidity is lower than said reference value. For example, the electronic control unit 50 can be configured to detect the humidity value present inside the environment A, measured by means of the hygrometric probe 45, and to compare this measured value with a reference value preset in the electronic control unit 50 thereof. If the measured value is lower than the reference value, then the electronic control unit 50 can be configured to activate the humidifier device 25A so as to raise the humidity value above this reference value and/or to generate an alarm signal (transmitted and emitted) by means of the interface 55.

Furthermore, the method can include measuring a value of a temperature inside the environment A, comparing the measured value with a threshold value, generating an alarm signal and/or lowering the temperature of the environment A if the measured temperature value is greater than said threshold value. For example, the electronic control unit 50 can be configured to detect the temperature value present inside the environment A, measured by means of the temperature probe 40, and to compare this measured value with a preset threshold value in the electronic control unit 50 thereof. If the measured value is greater than the threshold value, then the electronic control unit 50 can be configured to activate the conditioner device 25B so as to lower said temperature value below the threshold value and/or to generate an alarm signal (transmitted and emitted) by means of the interface 55.

The method can also include a preliminary step, to be carried out before the aforesaid steps, which includes determining, once the first value and a volume value of the environment A to be sanitized are known, if the first value can be reached; and to generate an alarm signal or in any case a signalling if the first value cannot be reached. For example, the electronic control unit 50 can, starting from the first value (preset in the electronic unit or possibly received as input by means of the interface 55), the volume value of the environment A to be sanitized (for example received as input by means of the interface) and from the quantity of ozone introduced in the time unit into the environment A (for example by means of the ozone generator device 15), implement a calculation algorithm (preset in the electronic control unit 50) which allows to determine the trend over time of the ozone concentration value. The electronic control unit 50, based on said trend over time of the ozone concentration value, can be configured to generate an alarm signal or in any case a signalling by means of the interface 55, if the trend over time does not allow to reach the first value or does not reach it within a pre-established maximum time.

In practice, ozone, as is known, naturally decays back to the state of oxygen in a relatively short time. Therefore, taking into account the spontaneous decay of ozone, the quantity of ozone introduced and the volume of the environment, the electronic control unit 50 can be configured in such a way as to check the possibility of reaching a desired ozone concentration value (e.g. the first value).

The invention thus conceived is susceptible to several modifications and variations, all falling within the scope of the inventive concept. Moreover, all details can be replaced by other technically equivalent elements. In practice, the materials used, as well as the contingent shapes and sizes, can be whatever according to the requirements without for this reason departing from the scope of protection of the following claims.

What is claimed is:

1. A method of decontaminating an environment, the method comprising:
measuring an ozone concentration value present inside the environment; and
carrying out a sequence of steps comprising:
(i) increasing the quantity of ozone present in the environment until the measured ozone concentration value reaches a first value,
(ii) lowering the quantity of ozone present in the environment until the measured ozone concentration value reaches a second value lower than the first value;
repeating the sequence of steps (i) and (ii) at least once such that the measured ozone concentration inside the environment sequentially reaches the first value, the second value, again the first value, and again the second value.

2. The method according to claim 1, wherein increasing the quantity of ozone comprises introducing ozone by means of an ozone generator device.

3. The method according to claim 1, wherein lowering the quantity of ozone comprises introducing ultraviolet rays into the environment.

4. The method according to claim 1, wherein lowering the quantity of ozone comprises converting the ozone in the environment into another substance by passing the ozone through a filter.

5. The method according to claim 4, wherein said filter is at least one of an activated carbon-based filter and a manganese oxide- and copper oxide-based filter.

6. The method according to claim 1, further comprising:
measuring a level of humidity present in the environment;
comparing the measured level of humidity with a reference value;
in response to the measured level of humidity being lower than said reference value, generating an alarm signal and/or humidifying the environment.

7. The method according to claim 1, further comprising:
measuring a value of a temperature inside the environment;
comparing the measured value with a threshold value;
in response to the measured temperature value being greater than said threshold value, generating an alarm signal and/or lowering the temperature of the environment.

8. The method according to claim 1, further comprising:
in response to the measured ozone concentration value being lower than said first value after a predetermined maximum time has lapsed from the beginning of said increasing the quantity of ozone step, generating an alarm signal and/or interrupting the increasing the quantity of ozone step.

9. The method according to claim 1, wherein the second value is in a range between ten percent (10%) of the first value and twenty-five percent (25%) of the first value.

10. A system for decontaminating an environment, the system comprising:
an ozone generator device configured to introduce ozone into the environment;
an ozone sensor configured to measure an ozone concentration value inside the environment; and
an electronic control unit operatively connected to the ozone sensor and to the ozone generator device, the electronic control unit being configured to selectively activate the ozone generator device to alternatingly (i) deactivate the ozone generator device when the ozone concentration value measured by the ozone sensor reaches a first value and (ii) activate the ozone generator device when the ozone concentration value measured by the ozone sensor reaches a second value lower than the first value.

11. A system according to claim 10, comprising a temperature sensor operatively connected to the electronic control unit and configured to measure a temperature value present in the environment.

12. A system according to claim 10, comprising a hygrometric sensor operatively connected to the electronic control unit and configured to measure a humidity value present in the environment.

13. The system according to claim 10, wherein the electronic unit is operatively connected to an interface and is configured to generate an alarm signal by means of said interface based upon a predetermined condition of the ozone concentration value measured by the ozone sensor.
